SEQUENCE LISTING (5 sequences)

SEQ ID NO: 1 - protein, 302 aa, Chlamydomonas reinhardtii
MSSDDYIKRGDLPTSKWSGRVTLRVDSAMAVPLDVVITYPSSGAAAYPVLVMYNGFQAKAPWYRGIVDHVSSWGYTVVQYTNGGLFPIVVDRVELTYLEPLLTWLETQSADAKSPLYGRADVSRLGTMGHSRGGKLAALQFAGRTDVSGCVLFDPVDGSPMTPESADYPSATKALAAAGRSAGLVGAAITGSCNPVGQNYPKFWGALAPGSWQMVLSQAGHMQFARTGNPFLDWSLDRLCGRGTMMSSDVITYSAAFTVAWFEGIFRPAQSQMGISNFKTWANTQVAARSITFDIKPMQSPQ

SEQ ID NO: 2 - protein, 262 aa, Arabidopsis thaliana
MGGEGGAEPVIHFVFVHGASHGAWCWYKLTTLLDAAGFKSTSVDLTGAGISLIDSNIVFDSDQYNRPLFSLLSDLPPHHKVILVGHSIGGGSVTEALCKFTDKISMAIYLAASMVQPGSIPSPHLSNIHVGEEDIWEYTYGEGTDKPPTGVLMKPEFIRHYYYSQSPLEDVTLSSKLLRPAPMRAFQDLDKLPPNPEAEKVPRVYIKTAKDNLFDSVRQDLLVENWPPSQLYVLEDSDHSAFFSVPTTLFAYLLRAVSFLQR

SEQ ID NO: 3 - protein, 526 aa, Chlamydomonas reinhardtii
MASAAPAVVPTSSTSADLDSQDGKYQWHSQWYPVGKVEDLDPRRPHATHLLGIPLALWKDAQGQWRAVEDRCSHRLAPLSEGRVEKDGTLQCAYHGWQFDGRGACTHIPQLRNDEKAQQVACASRRSCVRAYPTQVAHGLLWVLPDSSAAGWEKAATPPSTAQLLGGANDDGADPDVGWYVRDMPLRFDTLVENIADPSHVPFAHHGVQVCRLRAGPARTGRDSDHGRQGVAGLGWVQATSGRRTHPCLCAADHGRAVAASKPQGRRSQEKGTKLTLQDLGPDGFKTPFNMTGADMAVSFTAPSIVRYHPASSNTPFLILLAVPTRPGWSRLYHGLLNSGRKPTSFFFKILSAIRKIQWADHSFLRHPILDGDTYMLHVQERLLLGKNNDWKGSYYMPAPADGAVVGTRRWFDEHGGVVPTCEAGVAMPPQLSKRDAIERYHQHTKHCKVCSQALRNVETATWLAAAGGVLAALVLVARAVAGLPLLAPESVAPALAGAVCGGLVAVLQALRQQFIFVDYVHQDKN

SEQ ID NO: 4 - protein, 319 aa, Arabidopsis thaliana
MAMIFCNTLYSSSSPSYLSPLTSKPSRFSKNLRPRAQFQSMEDHDDHLRRKFMEFPYVSPTRKQLMVDLMSTVENRLQSQLLPCNLPPDVRNFNNPNGSAEASLHIRSGDKSSPIDFVIGSWIHCKIPTGVSLNITSISGFLNSSTKAPNFVVELIQSSSKSLVLILDLPHRKDLVLNPDYLKEYYQDTALDSHRQSLLKLPEVNPYVSPSLFVRSAFSPTASMLKIDAEEEDKLEEILRDHVSPAAKEVLEVWLERCVKEEEEKIVVGEEERMELERRDKSFRRKSIEDDLDLQFPRMFGEEVSSRVVHAIKEAFGVL

SEQ ID NO: 5 - protein, 322 aa, Chlamydomonas reinhardtii
MPSTQFLGASTLLLFGLRAVMSSDDYIKRGDLPTSKWSGRVTLRVDSAMAVPLDVVITYPSSGAAAYPVLVMYNGFQAKAPWYRGIVDHVSSWGYTVVQYTNGGLFPIVVDRVELTYLEPLLTWLETQSADAKSPLYGRADVSRLGTMGHSRGGKLAALQFAGRTDVSGCVLFDPVDGSPMTPESADYPSATKALAAAGRSAGLVGAAITGSCNPVGQNYPKFWGALAPGSWQMVLSQAGHMQFARTGNPFLDWSLDRLCGRGTMMSSDVITYSAAFTVAWFEGIFRPAQSQMGISNFKTWANTQVAARSITFDIKPMQSPQ

(19) United States
(12) Patent Application Publication
(10) Pub. No.: US 2017/0267984 A1
(43) Pub. Date: Sep. 21, 2017

(54) CHLOROPHYLLASE OVERPRODUCTION TO ENHANCE PHOTOSYNTHETIC EFFICIENCY

(71) Applicant: ExxonMobil Research and Engineering Company, Annandale, NJ (US)

(72) Inventors: Kelsey M. McNeely, Lambertville, NJ (US); Joseph C. Weissman, High Bridge, NJ (US)

(21) Appl. No.: 15/454,018

(22) Filed: Mar. 9, 2017

Related U.S. Application Data
(60) Provisional application No. 62/310,053, filed on Mar. 18, 2016.

(51) Int. Cl. C12N 9/18; C12P 7/64; C12N 9/02

(52) U.S. Cl. CPC C12N 9/18; C12Y 301/01014; C12N 9/0077; C12N 9/001; C12Y 301/01082; C12Y 114/15; C12P 7/6409; C12Y 103/07

(57) ABSTRACT

Recombinant microorganisms and methods for using the same are disclosed herein for producing biomass or at least one biomolecule. These methods comprise culturing a photosynthetic microorganism that can overexpress a chlorophyllase, and optionally isolating biomass and/or at least one biomolecule from the culture.

### CROSS-REFERENCE TO RELATED APPLICATIONS

This application claims priority to U.S. Provisional Application Ser. No. 62/310,053 filed on Mar. 18, 2016, herein incorporated by reference in its entirety.

### REFERENCE TO A SEQUENCE LISTING

This application contains references to amino acid sequences and/or nucleic acid sequences which have been submitted concurrently herewith as the sequence listing text file entitled “61691115_1.txt”, file size 15 KiloBytes (KB), created on 29 Feb. 2016. The aforementioned sequence listing is hereby incorporated by reference in its entirety pursuant to 37 C.F.R. §1.52(e)(5).

### FIELD

The present disclosure concerns recombinant microorganisms and methods for using the same to produce biomass or at least one biomolecule. These methods comprise culturing a photosynthetic microorganism that overexpresses a chlorophyllase, and isolating biomass or at least one biomolecule from the culture.

### BACKGROUND

Photosynthesis is the conversion of light energy to chemical energy by biological systems. Microalgae can be cultured photosynthetically for the production of various products, including proteins, peptides, amino acids, carbohydrates, terpenoids, isoprenoids, carotenoids, pigments, vitamins, bio-oils, and lipids, where light provides the energy for growth and biosynthesis of the algal products. Algae typically use only a percentage of the solar radiation incident on a pond surface, and photosynthesis can be inhibited by excess solar radiation. When photosynthetic microorganisms are exposed to light of an intensity that is greater than the capacity for photosynthetic utilization, as may occur at the upper level of a pond or the periphery of a photobioreactor culture, the photosynthetic microorganisms may engage mechanisms that dissipate light energy as heat to limit damage to the photosynthetic apparatus. This means that organisms on the light-facing periphery of the culture get more light than they can use, but organisms deeper inside the culture get little to no light at all. One possible way to allow photons to penetrate more deeply past the outermost layer of algal cells is to reduce the chlorophyll content of each cell. Melis (2009) Plant Sci. 177:272-80.

Chlorophyll a is a result of the chlorophyll biosynthetic pathway. Chlorophyll a can be either degraded via chlorophyllase or used as a precursor for chlorophyll b formation. See, e.g., FIG. 1 herein. Enzymatic degradation by chlorophyallase appears to be the rate limiting step for chlorophyll a catabolism in higher plants. Harpaz-Saad et al. (2007) Plant Cell 19:1007-22. Various genes encoding chlorophyllase have been identified and cloned. Tsuchiya et al. (1999) Proc. Nat'l. Acad. Sci. USA 96:15362-67. Chlorophyll production and light-harvesting antenna assembly are carefully regulated processes. When these processes are disturbed, toxic intermediates can form that damage the cell. Therefore, previous attempts to reduce antenna size by reducing chlorophyll content have met with only limited success.

### SUMMARY

A photosynthetic microorganism that overexpresses chlorophyllase is described herein. In certain embodiments, the photosynthetic microorganism also includes at least one non-native gene for the production of a biomolecule such as a protein, lipid, pigment, terpenoid, isoprenoid, carotenoid, vitamin, peptide, amino acid, and/or nucleotide. In certain embodiments, the cell that overexpresses chlorophyllase also overexpresses a magnesium dechelatase, a pheophorbidase, a pheophorbide a oxygenase, and/or a red chlorophyll catabolite reductase.

Methods of producing biomass or least one biomolecule are described herein. These methods comprise culturing a photosynthetic microorganism that overexpresses chlorophyllase under conditions sufficient for the microorganism to proliferate in the culture, and isolating biomass or at least one biomolecule from the culture. In some embodiments, the amount of biomass or a biomolecule produced by the culture is at least 10% greater (for example, at least 20% greater, at least 50% greater, at least 75% greater, at least 2-fold greater, or at least 10-fold greater) than the amount of biomass or a biomolecule produced by an identical culture of a microorganism identical in all respects except that it does not overexpress chlorophyllase. Additionally or alternatively, the photosynthetic microorganism can be cultured phototrophically and/or under intermittent light conditions, optionally including natural light. The photosynthetic microorganism can be cultured in a culture system that includes active mixing during at least a portion of the time the culture is exposed to light (the light period). For example, the photosynthetic microorganism can be cultured in a pond or photobioreactor.

### BRIEF DESCRIPTION OF THE DRAWINGS

FIG. 1 shows a set of metabolic pathways involving chlorophyll a.

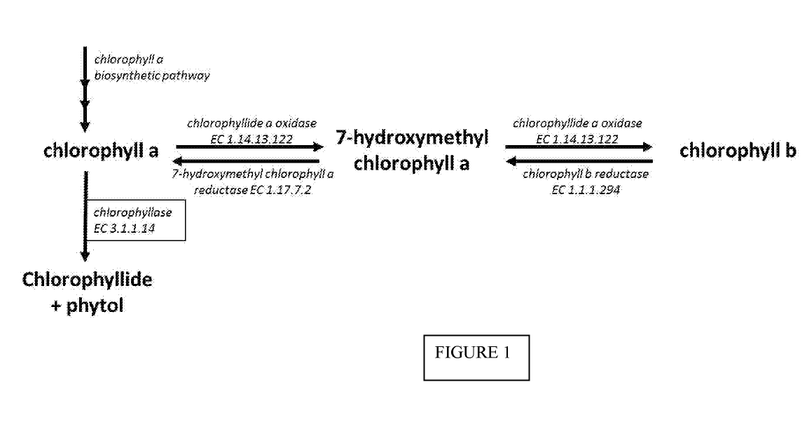

FIG. 2 shows an exemplary genetic construct for the overexpression of chlorophyllase. Solid lines indicate heterologous genes. Dashed lines indicate optional heterologous genes.

### DETAILED DESCRIPTION OF THE EMBODIMENTS

The present disclosure provides photosynthetic microorganisms that overexpress chlorophyllase and methods of producing biomass or at least one biomolecule comprising culturing a photosynthetic microorganism that overexpresses chlorophyllase.

### Glossary

Unless defined otherwise, all technical and scientific terms used herein have the same meaning as commonly understood by one of ordinary skill in the art to which this invention belongs. Unless otherwise required by context, singular terms shall include pluralities and plural terms shall include the singular. All publications, patents and other references mentioned herein are incorporated by reference in their entireties for all purposes as if each individual publication or patent application were specifically and individually indicated to be incorporated by reference. In case of conflict between definitions in the present text and those in the material incorporated by reference, the definitions in the present text will control over those incorporated by reference.

To “overexpress” chlorophyllase means that the photosynthetic organism in question produces quantitatively more chlorophyllase enzyme, which can be measured either by grams of cellular chlorophyllase or by chlorophyllase enzymatic activity, in a given unit of time, than is produced by a “wild-type” photosynthetic microorganism of the same species.

As used herein, when a comparison is made between a given microorganism and a corresponding “control” microorganism, the “control” microorganism is substantially identical to the given microorganism, except for a single modification in question. For example, if a given microorganism has been transduced to incorporate a heterologous chlorophyllase gene, then the “control” microorganism is the otherwise unmodified descendant of the transduced microorganism's parent cell. The “control” microorganism is substantially identical to the given microorganism to which a comparison is being made. In this context, “substantially” identical conveys that the control microorganism has not acquired any additional mutations that would materially affect the trait being compared between the control and given microorganisms. For example, if biomass accumulation is being compared between (1) a given microorganism that has been transduced with a heterologous chlorophyllase gene and (2) a corresponding control microorganism, the control microorganism cannot have suffered a frame-shift mutation in its own endogenous chlorophyllase gene that results in a prematurely truncated chlorophyllase enzyme. Such a mutation would be material, and therefore the control would not be substantially identical. However, the control microorganism could include an adventitious but silent mutation in its chlorophyllase gene, because such a mutation would not have a material effect on chlorophyllase production, and would therefore be insubstantial.

Illumination to drive photosynthesis can be “natural” light (e.g., direct sunlight, filtered sunlight, reflected sunlight, moonlight, etc.) or “artificial” light (e.g., incandescent electric light, fluorescent electric light, firelight, chemiluminescent illumination, etc.). “Artificial” light refers to light generated by human artifice (e.g., by lamps, candles, etc.), and “natural” light is all light that is not “artificial.”

A “biomolecule” refers to any organic molecule that is produced by a living organism, including large polymeric molecules. Biomolecules can be useful, for example, as fuels, fuel additives, or fuel precursors, including fuel feedstocks, as well as biomolecules that are useful as chemical, lubricants, surfactants, and/or detergents. A biomolecule produced using the methods described herein can be, without limitation, a protein, polymer, pigment, vitamin, peptide, amino acid, terpenoid, isoprenoid, and/or lipid (e.g., monoacylglyceride, diacylglyceride, triacylglyceride, fatty acid, fatty acid derivative, or the like). Advantageously, a biomolecule can be recovered from the culture, such as from the culture medium, the microorganism, or a combination thereof. In some preferred embodiments, a biomolecule produced using methods described herein can be a monoglyceride, diglyceride, triglyceride, free fatty acid, fatty acid derivative, or combination thereof.

“Biomass” refers to organic matter stored from plants and other living things and can be regarded as an energy source, including, but not limited to, an energy source that can be converted to fuel or fuel feedstocks. Alternatively or in addition, biomass can be recovered from the culture, and can optionally be used, for example, to extract, isolate, or purify one or more biomolecules or biomass components. Alternatively or in addition, biomass itself can be a product of the culture where the recovered biomass can be used in further processes for example, for producing heat, energy, nutrients, syngas, one or more alcohols, etc. or can be used as a food supplement or animal feed or supplement.

“Lipids” are a class of molecules that are typically soluble in nonpolar solvents (such as ether and chloroform) and are relatively or completely insoluble in water. Lipid molecules have these properties, because they consist largely of hydrocarbon tails which are hydrophobic in nature. Examples of lipids include fatty acids (saturated and unsaturated); glycerides or glycerolipids (such as monoglycerides (monoacylglycerides), diglycerides (diacylglycerides), triglycerides (triacylglycerides) or neutral fats, phosphoglycerides or glycerophospholipids, or the like, or combinations thereof); nonglycerides (such as sphingolipids, sterol lipids including cholesterol and steroid hormones, prenol lipids including terpenoids, isoprenoids, fatty alcohols, waxes, polyketides, or the like, or combinations thereof); and complex lipid derivatives (such as sugar-linked lipids, or glycolipids, protein-linked lipids, or the like, or a combination thereof). Fats are a subgroup of lipids and can include triacylglycerides.

“Lipases” are enzymes that catalyze the hydrolysis of ester bonds in glycerolipids, including, but not limited to, mono-, di-, and tri-acyl glycerols, as well as combinations thereof, to release free fatty acids and alcohols.

“Thioesterases” are enzymes that catalyze the cleavage of a fatty acid thioester. For example, “acyl-ACP thioesterase” is an enzyme that catalyzes the cleavage of a fatty acid from an acyl carrier protein (ACP). In some embodiments, the exogenous nucleic acid molecule encoding a thioesterase can include, without limitation, an acyl-ACP thioesterase, an acyl-CoA thioesterase, and a hydroxylbenzoyl-CoA thioesterase.

The term “gene” is used broadly to refer to any segment of nucleic acid (typically DNA, but optionally RNA) associated with expression of a given RNA or protein. Thus, genes include sequences encoding expressed RNA (which can include polypeptide coding sequences) and, often, the regulatory sequences required for their expression. Genes can be obtained from a variety of sources, including cloning from a source of interest or synthesizing from known or predicted sequence information and may include sequences designed to have desired parameters.

“Pfam” is a large collection of protein domains and protein families maintained by the Pfam Consortium and available at several sponsored world wide web sites. Pfam domains and families are identified using multiple sequence alignments and hidden Markov models (HMMs). Pfam-A families, which are based on high quality assignments, are generated by a curated seed alignment using representative members of a protein family and profile hidden Markov models based on the seed alignment, whereas Pfam-B families are generated automatically from the non-redundant clusters of the latest release of the Automated Domain Decomposition algorithm (ADDA; Heger A, Holm L (2003) J Mol Biol 328(3):749-67). All identified sequences belonging to the family are then used to automatically generate a full alignment for the familiy (Sonnhammer et al. (1998) Nucleic Acids Research 26: 320-322; Bateman et al. (2000) Nucleic Acids Research 26: 263-266; Bateman et al. (2004) Nucleic Acids Research 32, Database Issue: D138-D141; Finn et al. (2006) Nucleic Acids Research Database Issue 34: D247-251; Finn et al. (2010) Nucleic Acids Research Database Issue 38: D211-222). The phrase “conservative amino acid substitution” or “conservative mutation” refers to the replacement of one amino acid by another amino acid with a common property. A functional way to define common properties between individual amino acids is to analyze the normalized frequencies of amino acid changes between corresponding proteins of homologous organisms (Schulz, G. E. et al., (1979) Principles of Protein Structure, Springer-Verlag). According to such analyses, groups of amino acids can be defined where amino acids within a group exchange preferentially with each other, and therefore resemble each other most in their impact on the overall protein structure (Schulz, G. E. et al., (1979) Principles of Protein Structure, Springer-Verlag). Examples of amino acid groups defined in this manner can include: a “charged/polar group,” including Glu, Asp, Asn, Gln, Lys, Arg, and His; an “aromatic or cyclic group,” including Pro, Phe, Tyr, and Trp; and an “aliphatic group” including Gly, Ala, Val, Leu, Ile, Met, Ser, Thr, and Cys. Within each group, subgroups can also be identified. For example, the group of charged/polar amino acids can be sub-divided into sub-groups including: the “positively-charged sub-group,” comprising Lys, Arg and His; the “negatively-charged sub-group,” comprising Glu and Asp; and the “polar sub-group” comprising Asn and Gln. In another example, the aromatic or cyclic group can be sub-divided into sub-groups including: the “nitrogen ring sub-group,” comprising Pro, His, and Trp; and the “phenyl sub-group” comprising Phe and Tyr. In another further example, the aliphatic group can be sub-divided into sub-groups including: the “large aliphatic non-polar sub-group,” comprising Val, Leu, and Ile; the “aliphatic slightly-polar sub-group,” comprising Met, Ser, Thr, and Cys; and the “small-residue sub-group,” comprising Gly and Ala. Examples of conservative mutations include amino acid substitutions of amino acids within the sub-groups above, such as, but not limited to: Lys for Arg or vice versa, such that a positive charge can be maintained; Glu for Asp or vice versa, such that a negative charge can be maintained; Ser for Thr or vice versa, such that a free —OH can be maintained; and Gln for Asn such that a free —NH2 can be maintained.

A “conservative variant” of a polypeptide is a polypeptide having one or more conservative amino acid substitutions with respect to the reference polypeptide, in which the activity, substrate affinity, binding affinity of the polypeptide does not substantially differ from that of the reference polypeptide. A substitution, insertion, or deletion can be said to adversely affect the protein when the altered sequence substantially inhibits a biological function associated with the protein.

Percent identity or homology with respect to such sequences is defined herein as the percentage of amino acid residues in the candidate sequence that are identical with the known peptides, after aligning the sequences and introducing gaps, if necessary, to achieve the maximum percent homology and not considering any conservative substitutions as part of the sequence identity. N-terminal, C-terminal, and/or internal deletions and/or insertions into the peptide sequence shall not be construed as affecting homology.

Homology or identity at the nucleotide or amino acid sequence level as used herein are determined by BLAST (Basic Local Alignment Search Tool) analysis using the algorithm employed by the programs blastp, blastn, blastx, tblastn, and tblastx (Altschul et al. (1997), Nucleic Acids Res. 25, 3389-3402, and Karlin et al. (1990), Proc. Natl. Acad. Sci. USA 87, 2264-2268, both fully incorporated by reference), which are tailored for sequence similarity searching.

For blastn, designed for comparing nucleotide sequences, the scoring matrix is set by the ratios of M (i.e., the reward score for a pair of matching residues) to N (i.e., the penalty score for mismatching residues), wherein the default values for M and N can be +5 and −4, respectively. Four blastn parameters can be adjusted as follows: Q=10 (gap creation penalty); R=10 (gap extension penalty); wink=1 (generates word hits at every winkth position along the query); and gapw=16 (sets the window width within which gapped alignments are generated). The equivalent Blastp parameter settings for comparison of amino acid sequences can be: Q=9; R=2; wink=1; and gapw=32. A Bestfit comparison between sequences, available in the GCG package version 10.0, can use DNA parameters GAP=50 (gap creation penalty) and LEN=3 (gap extension penalty), and the equivalent settings in protein comparisons can be GAP=8 and LEN=2.

“Exogenous nucleic acid molecule” or “exogenous gene” refers to a nucleic acid molecule or gene that has been introduced (“transformed”) into a cell. A transformed cell may be referred to as a recombinant cell, into which additional exogenous gene(s) may be introduced. (A descendent of a cell that was transformed with a nucleic acid molecule is also referred to as “transformed” if it has inherited the exogenous nucleic acid molecule). The exogenous gene may be from a different species (and so “heterologous”), or from the same species (and so “homologous”), relative to the cell being transformed. An “endogenous” nucleic acid molecule, gene, or protein is the organism's own nucleic acid molecule, gene, or protein as it occurs in, or is naturally produced by, the organism. When a gene or a protein is “derived from” an organism, this should be understood to include sequences with at least 60% (e.g., at least 65%, at least 70%, at least 75%, at least 80%, at least 85%, at least 90%, at least 95%, at least 96%, at least 97%, at least 98%, at least 90%, or even 100%) primary sequence identity to the wild-type gene or protein isolated from the organism in question.

When referring to a gene regulatory sequence or to an auxiliary nucleic acid sequence used for maintaining or manipulating a gene sequence (e.g., a 5′ untranslated region, 3′ untranslated region, poly A addition sequence, intron sequence, splice site, ribosome binding site, internal ribosome entry sequence, genome homology region, recombination site, etc.), “heterologous” means that the regulatory sequence or auxiliary sequence is from a different source than the gene with which the regulatory or auxiliary nucleic acid sequence is juxtaposed in a construct, genome, chromosome, or episome. Thus, a promoter operably linked to a gene to which it is not operably linked to in its natural state (i.e., in the genome of a non-genetically engineered organism) is referred to herein as a “heterologous promoter”, even though the promoter may be derived from the same species (or, in some cases, the same organism) as the gene to which it is linked.

The term “native” is used herein to refer to nucleic acid sequences or amino acid sequences as they naturally occur in the host. The term “non-native” is used herein to refer to nucleic acid sequences or amino acid sequences that do not occur naturally in the host. A nucleic acid sequence or amino acid sequence that has been removed from a host cell, subjected to laboratory manipulation such that it no longer contains the same sequence as when it was removed, and reintroduced into the host cell is considered “non-native.” Synthetic or partially synthetic genes introduced into a host cell are “non-native.” Non-native genes further include genes endogenous to the host microorganism operably linked to one or more heterologous regulatory sequences that have been recombined into the host genome.

A “recombinant” or “engineered” nucleic acid molecule is a nucleic acid molecule that has been altered through human manipulation. As non-limiting examples, a recombinant nucleic acid molecule includes any nucleic acid molecule that: 1) includes conjoined nucleotide sequences that are not conjoined in nature, 2) has been engineered using molecular cloning techniques such that it lacks one or more nucleotides with respect to the naturally occurring nucleic acid molecule sequence, and/or 3) has been manipulated using molecular cloning techniques such that it has one or more sequence changes or rearrangements with respect to the naturally occurring nucleic acid sequence. As non-limiting examples, a cDNA lacking at least one intron sequence that would be found in the corresponding gDNA sequence is a recombinant DNA molecule, as is any nucleic acid molecule to which linkers have been attached, or that has been integrated into a vector, such as a cloning vector or expression vector.

When applied to organisms, the term recombinant, engineered, or genetically engineered refers to organisms that have been manipulated by introduction of an exogenous or recombinant nucleic acid sequence into the organism, and includes organisms having gene knockouts, targeted mutations and gene replacement, promoter replacement, deletion, or insertion, as well as organisms having exogenous genes that have been introduced into the organism. An exogenous or recombinant nucleic acid molecule can be integrated into the recombinant/genetically engineered organism's genome or in other instances may not be integrated into the recombinant/genetically engineered organism's genome.

The term “recombinant protein” as used herein refers to a protein produced by genetic engineering.

An “expression cassette”, as used herein, refers to a gene encoding a protein or functional RNA (e.g., a tRNA, a microRNAs, a ribosomal RNA, etc.) operably linked to expression control sequences, such as a promoter, and optionally, any or a combination of other nucleic acid sequences that affect the transcription or translation of the gene, such as, but not limited to, a transcriptional terminator, a ribosome binding site, a splice site or splicing recognition sequence, an intron, an enhancer, a polyadenylation signal, an internal ribosome entry site, etc.

When referring to a gene regulatory sequence or to an auxiliary nucleic acid sequence used for maintaining or manipulating a gene sequence (e.g., a 5′ untranslated region, 3′ untranslated region, poly A addition sequence, intron sequence, splice site, ribosome binding site, internal ribosome entry sequence, genome homology region, recombination site, etc.), “heterologous” means that the regulatory sequence or auxiliary sequence is from a different source than the gene with which the regulatory or auxiliary nucleic acid sequence is juxtaposed in a construct, genome, chromosome, or episome. Thus, a promoter operably linked to a gene to which it is not operably linked to in its natural state (i.e., in the genome of a non-genetically engineered organism) is referred to herein as a “heterologous promoter”, even though the promoter may be derived from the same species (or, in some cases, the same organism) as the gene to which it is linked.

As used herein “attenuated” means reduced in amount, degree, intensity, or strength. Attenuated gene expression may refer to a significantly reduced amount and/or rate of transcription of the gene in question, or of translation, folding, or assembly of the encoded protein.

A “free fatty acid”, as used herein, is a non-esterified acyl moiety that is substantially unassociated, e.g., with an enzyme and/or protein, within or outside an organism (e.g., globular and/or micellular storage within an organism, without esterification, can still qualify as a free fatty acid). Thus, a free fatty acid need not necessarily be a strict acid or be structurally “free”, but a free fatty acid specifically does not include an acyl moiety whose carboxylate oxygen is covalently linked to any other moiety other than a hydrogen atom, meaning that fatty acid esters are specifically not included in free fatty acids. However, a free fatty acid can advantageously include an acyl moiety containing at least four carbons (preferably at least 6 carbons, for example at least 8 carbons), in which the acyl moiety (i) is covalently linked to a hydrogen atom, (ii) has an ionic charge, to which a counterion can be associated (even if loosely and/or solvent-separated), and/or (iii) is associated, but not covalently bonded to another moiety that is relatively easily transformable into the corresponding acid form or the corresponding ionic form (e.g., through hydrogen-bonding or the like). Nonlimiting examples of counterions can include metals salts (such as calcium, magnesium, sodium, potassium, aluminum, iron, and the like, and combinations thereof), other inorganic ions (such as ammonium, mono-, di-, tri-, and tetra-alkylammonium, sulfonium, phosphonium, and the like, and combinations thereof), organic ions (such as carbocations), and the like, and combinations thereof.

“Expression vector” or “expression construct” refers to a nucleic acid that has been generated via human intervention, including by recombinant means and/or direct chemical synthesis, with a series of specified nucleic acid elements that permit transcription and/or translation of a particular nucleic acid in a host cell. The expression vector can be a plasmid, a part of a plasmid, a viral construct, a nucleic acid fragment, or the like, or a combination thereof. Typically, the expression vector can include a nucleic acid to be transcribed operably linked to a promoter in an “expression cassette.” “Inducible promoter” refers a promoter that mediates transcription of an operably linked gene in response to a particular stimulus. “Operable linkage” is a functional linkage between two nucleic acid sequences, such as a control sequence (typically a promoter) and the linked sequence (typically a sequence that encodes a protein and/or other biomolecule, also called a coding sequence). A promoter is in operable linkage with an exogenous gene if it can mediate transcription of the gene.

Vectors can be introduced into prokaryotic and eukaryotic cells via conventional transformation and/or transfection techniques. The terms “transformation,” “transfection,” “conjugation,” and “transduction,” as used in the present context, are intended to comprise a multiplicity of methods known to those skilled in the art for the introduction of foreign nucleic acid (e.g., exogenous DNA) into a host cell. Such methods including calcium phosphate and/or calcium chloride co-precipitation, DEAE-dextran-mediated transfection, lipofection, natural competence, chemically mediated transfer, electroporation, particle bombardment, or the like, or combinations thereof. Examples of suitable methods for the transformation and/or transfection of host cells, e.g., can be found in Molecular Cloning—A Laboratory Manual (2001), Cold Spring Harbor Laboratory Press.

### Microorganisms

Photosynthetic microorganisms, as described herein, can overexpress chlorophyllase. In certain embodiments, the overexpressing microorganism can contain more copies of a chlorophyllase gene than the corresponding control microorganism. In certain embodiments, the overexpressing and control microorganisms can contain the same number of chlorophyllase genes, but the overexpressing microorganism can use different and more active promoters to drive gene expression, or can include additional sequences to stabilize chlorophyllase mRNA and otherwise enhance chlorophyllase message translation. In still other embodiments, the overexpressing microorganism can add and/or delete other factors in the cell, e.g., to reduce turnover of chlorophyllase protein and otherwise stabilize chlorophyllase, so as to increase the equilibrium cellular content of enzymatically active chlorophyllase relative to the content of the control microorganism.

The genetically engineered, for example recombinant, microorganism can be any photosynthetic microorganism, including without limitation, a cyanobacterium, a eukaryotic microalga, or the like. The microorganisms according to some embodiments can include, but are not limited to, the following genera of cyanobacteria: Agmenellum, Anabaena, Anabaenopsis, Anacystis, Aphanizomenon, Arthrospira, Asterocapsa, Borzia, Calothrix, Chamaesiphon, Chlorogloeopsis, Chroococcidiopsis, Chroococcus, Crinalium, Crocosphaera, Cyanobacterium, Cyanobium, Cyanocystis, Cyanospira, Cyanothece, Cylindrospermopsis, Cylindrospermum, Dactylococcopsis, Dermocarpella, Fischerella, Fremyella, Geitleria, Geitlerinema, Gloeobacter, Gloeocapsa, Gloeothece, Halospirulina, Iyengariella, Leptolyngbya, Limnothrix, Lyngbya, Microcoleus, Microcystis, Myxosarcina, Nodularia, Nostoc, Nostochopsis, Oscillatoria, Phormidium, Planktothrix, Pleurocapsa, Prochlorococcus, Prochloron, Prochlorothrix, Pseudanabaena, Rivularia, Schizothrix, Scytonema, Spirulina, Stanieria, Starria, Stigonema, Symploca, Synechococcus, Synechocystis, Thermosynechococcus, Tolypothrix, Trichodesmium, Tychonema, and Xenococcus. The microorganisms according to some embodiments can include, but are not limited to, the following genera of a microalgae: Achnanthes, Amphiprora, Amphora, Ankistrodesmus, Asteromonas, Boekelovia, Bolidomonas, Borodinella, Botrydium, Botryococcus, Bracteococcus, Chaetoceros, Carteria, Chlamydomonas, Chlorococcum, Chlorogonium, Chlorella, Chroomonas, Chrysosphaera, Cricosphaera, Crypthecodinium, Cryptomonas, Cyclotella, Dunaliella, Elhpsoidon, Emiliania, Eremosphaera, Ernodesmius, Euglena, Eustigmatos, Franceia, Fragilaria, Gloeothamnion, Haematococcus, Halocafeteria, Heterosigma, Hymenomonas, Isochrysis, Lepocinclis, Micractinium, Monoraphidium, Nannochloris, Nannochloropsis, Navicula, Neochloris, Nephrochloris, Nephroselmis, Nitzschia, Ochromonas, Oedogonium, Oocystis, Ostreococcus, Pavlova, Parachlorella, Pascheria, Pelagomonas, Phaeodactylum, Phagus, Picochlorum, Platymonas, Pleurochrysis, Pleurococcus, Prototheca, Pseudochlorella, Pseudoneochloris, Pseudostaurastrum, Pyramimonas, Pyrobotrys, Scenedesmus, Schizochlamydella, Skeletonema, Spyrogyra, Stichococcus, Tetrachlorella, Tetraselmis, Thalassiosira, Tribonema, Vaucheria, Viridiella, Vischeria, and Volvox. For example, the microorganism can be of the genus Chlamydomonas, Nannochloropsis, or Tetraselmis. In certain embodiments, the photosynthetic microorganism is (a) a microorganism as described in U.S. Pat. No. 8,940,508 or in U.S. Ser. No. 14/099,879; or (b) a microorganism containing a genetic modification as described in U.S. Pat. No. 8,940,508 or in U.S. Ser. No. 14/099,879. Both U.S. Pat. No. 8,940,508 and U.S. Ser. No. 14/099,879 are herein incorporated by reference in their entireties.

In certain embodiments, the chlorophyllase can be a chlorophyllase isolated or derived from a higher plant, such as Citrus sinensis, or from a eukaryotic alga, such as Chlamydomonas reinhardtii. For example, in certain embodiments, the chlorophyllase can have at least 50%, at least 55%, at least 60%, at least 65%, at least 70%, at least 75%, at least 80%, at least 85%, at least 90%, at least 95%, at least 96%, at least 97%, at least 98%, at least 99%, or even 100% amino acid sequence identity to SEQ ID NO:1. In certain embodiments, the chlorophyllase can have between 85% and 95% sequence identity to SEQ ID NO:1.

Photosynthetic microorganisms overexpressing chlorophyllase can have reduced chlorophyll a content, relative to the corresponding control microorganism. In certain embodiments, the chlorophyll content of the microorganisms overexpressing chlorophyllase can have at least 5%, at least 10%, at least 15%, at least 20%, at least 30%, at least 40%, at least 50%, at least 60%, at least 70%, at least 80%, or at least 90% reduction in chlorophyll content, relative to a corresponding control microorganism. Additionally or alternatively, the microorganism overexpressing chlorophyllase can have a chlorophyll content reduction no more than 80%, no more than 70%, no more than 60%, no more than 50%, no more than 40%, no more than 30%, no more than 20%, no more than 15%, no more than 10%, no more than 5%, no more than 4%, no more than 3%, no more than 2%, or no more than 1%, relative to a corresponding control microorganism. Reduced chlorophyll content can, in turn, result in smaller light-harvesting antennæ.

Photosynthetic microorganisms overexpressing chlorophyllase can unexpectedly exhibit higher productivity than corresponding control microorganisms. Without limiting the invention to any particular mechanism, it is contemplated that, in a production system in which cultures are mixed for optimal exposure to light, CO₂, and nutrients, the light environment for individual cells can change quickly as mixing occurs. Because the chlorophyllase-overexpressing microorganisms can have smaller light-harvesting antennæ, those microorganisms at the edge of the culture—where light intensity is greatest—can absorb fewer photons, thus allowing more photons to penetrate deeper into the culture to be used by other microorganisms.

### Further Modified Microorganisms

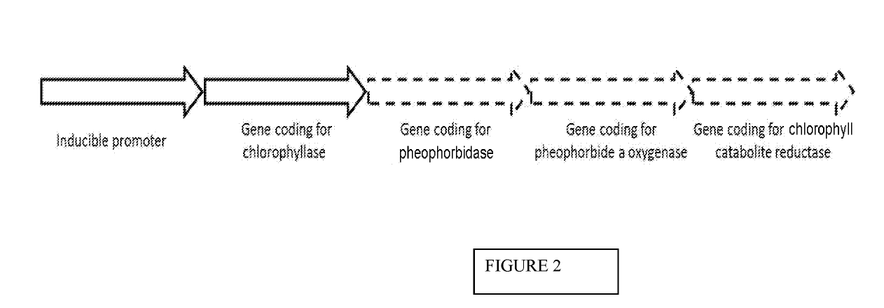

Also described herein are cultures of recombinant photosynthetic microorganisms that can express (or overexpress) one or more additional enzymes selected from the group consisting of (1) magnesium dechelatase, (2) pheophorbide a oxygenase, (3) pheophorbidase, and (4) red chlorophyll catabolite reductase. In certain embodiments, the photosynthetic microorganism can overexpress chlorophyllase in combination with: (1) & (2); (1) & (3); (1) & (4); (2) & (3); (2) & (4); (3) & (4); (1) & (2) & (3); (1) & (2) & (4); (1) & (3) & (4); (2) & (3) & (4); or (1) & (2) & (3) & (4). In certain embodiments, the nucleotide sequence encoding the magnesium dechelatase, the pheophorbide a oxygenase, the pheophorbidase, and/or the red chlorophyll catabolite reductase can be operably linked to the nucleotide sequence encoding the chlorophyllase, such that the various operably linked nucleotide sequences can be expressed under the control of the same promoter. See, e.g., FIG. 2 herein.

In certain embodiments, the pheophorbidase can be a pheophorbidase isolated or derived from a eukaryotic alga or from a higher plant, such as Arabidopsis thaliana. For example, in certain embodiments, the pheophorbidase can have at least 50%, at least 55%, at least 60%, at least 65%, at least 70%, at least 75%, at least 80%, at least 85%, at least 90%, at least 95%, at least 96%, at least 97%, at least 98%, at least 99%, or as much as 100% amino acid sequence identity to SEQ ID NO:2. In certain embodiments, the pheophorbidase can have between 85% and 95% sequence identity to SEQ ID NO:2.

In certain embodiments, the pheophorbide a oxygenase can be a pheophorbide a oxygenase isolated or derived from a higher plant or from a eukaryotic alga, such as Chlamydomonas reinhardtii. For example, in certain embodiments, the pheophorbide a oxygenase can have at least 50%, at least 55%, at least 60%, at least 65%, at least 70%, at least 75%, at least 80%, at least 85%, at least 90%, at least 95%, at least 96%, at least 97%, at least 98%, at least 99%, or as much as 100% amino acid sequence identity to SEQ ID NO:3. In certain embodiments, the pheophorbide a oxygenase can have between 85% and 95% sequence identity to SEQ ID NO:3.

In certain embodiments, the red chlorophyll catabolite reductase can be a red chlorophyll catabolite reductase isolated or derived from a eukaryotic alga or from a higher plant, such as Arabidopsis thaliana (for example, A. thaliana ACD2). For example, in certain embodiments, the red chlorophyll catabolite reductase can have at least 50%, at least 55%, at least 60%, at least 65%, at least 70%, at least 75%, at least 80%, at least 85%, at least 90%, at least 95%, at least 96%, at least 97%, at least 98%, at least 99%, or as much as 100% amino acid sequence identity to SEQ ID NO:4. In certain embodiments, the red chlorophyll catabolite reductase can have between 85% and 95% sequence identity to SEQ ID NO:4.

Other Modifications for Producing Free Fatty Acids and/or Fatty Acid Derivatives

In certain embodiments, the photosynthetic microorganisms that overexpress a chlorophyllase can further include additional genetic modifications that enhance the productivity or robustness of the strain. For example, one or more recombinant nucleic acid molecules can be introduced into the microorganisms for directing the production of particular biomolecules, and/or for increasing or decreasing expression of endogenous genes that can directly/indirectly enhance the production of biomass in general and/or of particular biomolecules in the modified strain. For example, an expression system for one or more exogenous genes, such as thioesterase and lipase genes, may be introduced into the microorganism overexpressing a chlorophyllase. For example, the photosynthetic microorganism overexpressing a chlorophyllase can, in some embodiments, comprise one or more recombinant nucleic acid molecules encoding a thioesterase and/or a polypeptide having lipolytic activity. Useful thioesterases and lipolytic enzymes can include heterologous thioesterase and/or lipase genes capable of producing free fatty acids from membrane lipids or storage lipids, e.g., phospholipids, triacylglycerol, diacylglycerol, monoacylglycerol, or the like, or combinations thereof. For example, a recombinant or exogenous nucleic acid molecule can encode one or more of the following: an acyl-ACP thioesterase; an acyl-CoA thioesterase; a hydroxybenzoyl-CoA thioesterase; a lipase that is a member of a Pfam belonging to the AB Hydrolase Pfam clan (CL0028); a lipase including a LipA domain identified as conserved protein domain COG1075, or included in the protein family Pfam PF01674; a lipase including a Lipase 3 domain identified as conserved protein domain COG3675, or included in the protein family Pfam PF01764; a lipase included in the protein family Pfam PF07819; a lipase included in the protein family Pfam PF03583; a lipase included in the protein family Pfam PF00151; and a polypeptide having lipolytic activity that recruits to Pfam PF00561, Pfam PF02230, Pfam PF07859, Pfam PF08386, Pfam PF12695, Pfam PF12697, Pfam PF12715, Pfam PF04083, and/or Pfam PF01425. A microorganism for the production of free fatty acids, in some embodiments, can be transformed with a gene encoding an exogenous acyl-ACP thioesterase, such as a gene encoding a polypeptide that when queried against the Pfam database, provides a match with Pfam PF01643 having a bit score of less than or equal to 20.3 (the gathering cut-off for PF01643). The exogenous acyl-ACP thioesterase gene can encode an acyl-ACP thioesterase from a higher plant species. Genes encoding acyl-ACP thioesterases derived from higher plants can include, without limitation, genes encoding acyl-ACP thioesterases from Cuphea species (e.g., Cuphea carthagenensis, Cuphea wrightii (e.g., AAC49784.1 GI:1336008), Cuphea lanceolata (e.g, CAA54060, GI495227), Cuphea palustris, (e.g., AAC49783.1 GI:1336006; AAC49179.1 GI:1215718); Cuphea hookeriana (e.g., AAC72882.1 GI:3859830; AAC49269.1 GI:1292906; AAC72881.1 GI:3859828; AAC72883.1 GI:3859832), Cuphea calophylla (e.g., ABB71580.1 GI:81361963)) or genes from other higher plant species. For example, a microorganism used in the methods and cultures disclosed herein can include a gene encoding an acyl-ACP thioesterase from species such as but not limited to, Arabidopsis (XP_002885681.1 GI:297835598; NP_172327.1 GI:15223236); Arachis hypogaea (e.g., AB038556.1 GI:133754634); Brassica species (e.g., CAA52069.1 GI:435011), Camellia oleifera ((e.g., ACQ57189.1 GI:229358082); Cinnamonum camphorum (e.g., AAC49151.1 GI:1143156); Cocos nucifera; Glycine max (e.g., ABD91726.1 GI:90192131); Garcinia mangostana (e.g., AAB51525.1 GI:1930081); Gossypium hirsutum (e.g., AAD01982.1 GI:4104242); Helianthus annuus (e.g., AAQ08226 GI:33325244); Jatropha curcas (e.g., ABU96744.1 GI:156900676); Macadamia tetraphylla (e.g., ADA79524.1 GI:282160399); Elaeis oleifera (e.g., AAM09524.1 GI:20067070); Oryza sativa (e.g., BAA83582.1 GI:5803272); Populus tomentosa (e.g., ABC47311.1 GI:83778888); Umbellularia californica (e.g., AAC49001.1 GI:595955); Ulmus Americana (e.g., AAB71731.1 GI:2459533); and Zea mays (ACG41291.1 GI:195643646), or any of those disclosed in U.S. Pat. No. 5,455,167; U.S. Pat. No. 5,654,495; and U.S. Pat. No. 5,455,167; all incorporated by reference herein in their entireties. Additional or alternative examples can include acyl-ACP thioesterases from mosses (Bryophyta), such as, for example, Physcomitrella patens, (e.g., XP_001770108.1 GI:168035219). These examples are not meant to be limiting with regard to the types or specific examples of acyl-ACP thioesterase genes that can be used. Further examples can include acyl-ACP thioesterase genes from additional organisms, including, for example, prokaryotic organisms. Illustrative examples of prokaryotic acyl-ACP thioesterases that may be expressed by a microorganism useful in the methods and cultures provided herein include, but are not limited to, acyl-ACP thioesterases from Desulfovibrio desulfuricans (e.g., Q312L1 GI:123552742); Elusimicrobium minutum (e.g., ACC98705 GI:186971720); Carboxydothermus hydrogenoformans (e.g., YP_359670 GI:78042959); Clostridium thermocellum (e.g., YP_001039461 GI:125975551); Moorella thermoacetica (e.g., YP_431036 GI:83591027); Geobacter metallireducens (e.g., YP_384688 GI:78222941); Salinibacter ruber (e.g., YP_444210 GI:83814393); Microscilla marina (e.g., EAY28464 123988858); Parabacteroides distasonis (e.g., YP_001303423 GI:150008680); Enterococcus faecalis (e.g., ZP_03949391 GI:227519342); Lactobacillus plantarum (e.g., YP_003062170 GI:254555753); Leuconostoc mesenteroides (e.g., YP_817783 GI:116617412); Oenococcus oeni (e.g., ZP_01544069 GI:118586629); Mycobacterium smegmatis (e.g., ABK74560 GI:118173664); Mycobacterium vanbaalenii (e.g., ABM11638 GI:119954633); Rhodococcus erythropolis (e.g., ZP_04385507 GI:229491686; Rhodococcus opacus (e.g., YP_002778825 GI:226361047), or any of those disclosed in U.S. Pat. No. 8,530,207, which is incorporated herein by reference in its entirety. Still further additionally or alternatively, the microorganism can include nucleic acid molecules encoding variants of the above-listed acyl-ACP thioesterases, acyl-CoA thioesterases, hydroxylbenzoyl-CoA thioesterases, or lipases, in which the variants can have at least 80%, for example at least 85%, at least 90%, or at least 95%, identity to the amino acid sequences accessed by the provided or referenced Genbank Accession Numbers, and in which the variants have at least the level of activity (e.g., thioesterase or lipase activity) as the reference sequence.

Further additionally or alternately, the recombinant microorganism can include those genetically engineered with exogenous and/or endogenous genes encoding polypeptides having lipolytic activity, such as, for example, lipases/esterases capable of producing free fatty acids from membrane lipids or storage lipids, e.g., phospholipids, glycolipids, triacylglycerols, diacylglycerols, monoacylglycerols, or the like, or combinations thereof. Lipases are enzymes that catalyze the hydrolysis of ester bonds in glycerolipids, including, but not limited to, mono-, di-, and tri-acyl glycerols, as well as combinations thereof, to release free fatty acids and alcohols.

The use of lipase genes in microorganisms used in the production of free fatty acids is disclosed in U.S. Pat. No. 9,175,256, which is incorporated herein by reference in its entirety. The lipase gene can be a gene encoding any lipase, e.g., that can liberate a fatty acid from a glycerolipid (including a monoglyceride, a diglyceride, a triglyceride, a phospholipid, a galactolipid, etc.). For example, a lipase gene can encode a polypeptide having lipase activity that is a member of the Pfam AB Hydrolase clan, CL0028, such as but not limited to, a lipase that is a member of Pfam 01674, Pfam 01764, Pfam 07819, Pfam 03583, and/or Pfam 00151. In some embodiments, an exogenous lipase gene introduced into a microorganism can encode a protein with an amino acid sequence having an E-value parameter of 0.01 or less when queried using the Pfam Profile HMM for any of Pfam PF01674, Pfam PF 01764, Pfam PF07819, Pfam PF03583, and/or Pfam PF00151. Additionally or alternatively contemplated are recombinant microorganisms engineered to include gene regulatory sequences that can induce or increase expression of an endogenous lipase gene. For example, a microorganism can be engineered such that a heterologous promoter is inserted upstream of a coding region of an endogenous lipase gene. The heterologous promoter can replace an endogenous promoter and/or can be inserted upstream or downstream of the endogenous promoter that regulates expression of the endogenous lipase gene, for example using homologous recombination or site-specific recombination. The heterologous promoter can be a constitutive promoter or an inducible promoter increasing expression of the endogenous lipase gene.

A photosynthetic microorganism overexpressing chlorophyllase including a recombinant gene encoding a protein that participates in the production of fatty acids, such as, for example, a recombinant thioesterase and/or lipase gene, can produce at least one free fatty acid, such as one or more of a C₆, C₈, C₁₀, C₁₂, C₁₄, C₁₆, C₁₈, C₂₀, C₂₂, and C₂₄ free fatty acid. In some embodiments, the microorganism can produce at least one free fatty acid during the growth of the culture, and/or can produce at least one free fatty acid in the absence of disruption or lysis of the cells. Recombinant thioesterase and/or lipase genes can optionally additionally be introduced into such photosynthetic microorganisms overexpressing chlorophyllase, e.g., for the production of fatty acid derivatives.

Additionally or alternatively, microorganisms overexpressing a chlorophyllase can also comprise at least one additional exogenous nucleic acid molecule that encodes a polypeptide that can participate in the synthesis of a fatty acid. For example, a transgenic microorganism for the production of one or more fatty acids can include an exogenous gene encoding an acetyl-CoA carboxylase, a malonyl CoA:ACP transacylase, or a beta-ketoacyl-ACP synthase, or a combination thereof. Further additionally or alternatively, a photosynthetic microorganism overexpressing chlorophyllase can include one or more recombinant nucleic acid molecules encoding a protein that can participate in the production of a fatty acid derivative, for example, one or more recombinant nucleic acid molecules encoding any combination of an acyl-CoA reductase, a carboxylic acid reductase, an acyl-ACP reductase, a fatty aldehyde reductase, a wax synthase, a fatty acid decarboxylase, a fatty aldehyde decarbonylase, and/or an acyl-CoA synthetase.

The photosynthetic microorganisms described herein may further include at least one endogenous gene that is attenuated or disrupted. Such an endogenous gene that can be attenuated or disrupted in the recombinant microorganism can include, but is not limited to, one or more genes encoding inactivated and/or downregulated beta-oxidation pathway enzymes, and/or the enzymes themselves, which are operative on such beta-oxidation pathways, may be inhibited. This could prevent the degradation of fatty acids released from acyl-ACPs, thus enhancing the yield of secreted fatty acids. In cases where the desired products are medium-chain fatty acids, the inactivation and/or downregulation of genes that encode acyl-CoA synthetase and/or acyl-CoA oxidase enzymes that preferentially use these chain lengths as substrates could be beneficial. Mutations in the genes encoding medium-chain-specific acyl-CoA synthetase and/or medium-chain-specific acyl-CoA oxidase enzymes, such that the activity of the enzymes could be diminished, may additionally or alternatively be effective in increasing the yield of produced and/or released fatty acids. An additional modification can inactivate and/or downregulate the acyl-ACP synthetase gene and/or can inactivate and/or inhibit the encoded protein. Mutations in the genes can be introduced either by recombinant or non-recombinant methods. These enzymes and their genes are known and may be targeted specifically by disruption, deletion, generation of antisense sequences, generation of ribozymes, and/or other recombinant approaches known to the practitioner. Inactivation of the genes can additionally or alternately be accomplished by random mutation techniques such as exposure to UV and/or chemical mutagens, and the resulting cells can be screened for successful mutants. The proteins themselves can be inhibited by intracellular generation of appropriate antibodies, intracellular generation of peptide inhibitors, or the like, or some combination thereof.

Still further additionally or alternatively, the photosynthetic microorganism can be modified such that one or more genes encoding storage carbohydrate and/or polyhydroxyalkanoate (PHA) biosynthesis pathway enzymes can be inactivated and/or downregulated, and/or such that the enzymes themselves, which are operative on such pathways, can be inhibited. Examples include, but not limited to, enzymes involved in glycogen, starch, or chrysolaminarin synthesis, including glucan synthases and branching enzymes. Other examples include enzymes involved in PHA biosynthesis, such as acetoacetyl-CoA synthase and PHA synthase.

### Further Modifications for Producing Fatty Acid Derivatives

Additionally or alternatively, to providing an expression system for one or more appropriate recombinant genes, such as lipase genes, further modifications in the microorganism may be made. For example, in some embodiments, the genetically engineered photosynthetic microorganism overexpressing chlorophyllase can produce a fatty aldehyde and can include one or more nucleic acid molecules encoding an exogenous acyl-CoA reductase, carboxylic acid reductase, and/or acyl-ACP reductase. Additionally or alternatively, the genetically engineered photosynthetic microorganism can produce a fatty alcohol and can include (i) at least one nucleic acid molecule encoding an exogenous acyl-CoA reductase, carboxylic acid reductase, and/or acyl-ACP reductase; and/or (ii) at least one exogenous fatty aldehyde reductase. Alternatively or in addition, the genetically engineered photosynthetic microorganism described herein can produce a wax ester and can include (iii) one or more nucleic acid molecules encoding an exogenous acyl-CoA reductase, carboxylic acid reductase, and/or acyl-ACP reductase; and (iv) an exogenous wax synthase. Wax esters include an A chain and a B chain linked through an ester bond, one or both of which can be derived from a fatty acid generated by the exogenous 4-hydroxybenzoyl-CoA thioesterase. Wax esters produced by a photosynthetic microorganism including a nucleic acid molecule encoding an exogenous 4-hydroxybenzoyl-CoA thioesterase therefore can have A+B chain lengths of, for example, 16 to 36 carbons, 16 to 32 carbons, or 24 to 32 carbons.

Further additionally or alternatively, a genetically engineered photosynthetic microorganism that produces a fatty alcohol, fatty aldehyde, wax ester, alkane, and/or alkene may optionally include a nucleic acid molecule encoding an acyl-CoA synthetase.

### Expression Systems

In some embodiments, recombinant microorganisms as described herein can be transformed with exogenous genes by the introduction of appropriate expression vectors.

For example, algae and photosynthetic bacteria can be transformed by any suitable methods, including, as non-limiting examples, natural DNA uptake (Chung et al. (1998) FEMS Microbiol. Lett. 164: 353-361; Frigaard et al. (2004) Methods Mol. Biol. 274: 325-40; Zang et al. (2007) J. Microbiol. 45: 241-245), conjugation, transduction, glass bead transformation (Kindle et al. (1989) J. Cell Biol. 109: 2589-601; Feng et al. (2009) Mol. Biol. Rep. 36: 1433-9; U.S. Pat. No. 5,661,017), silicon carbide whisker transformation (Dunahay et al. (1997) Methods Mol. Biol. 62: 503-9), biolistics (Dawson et al. (1997) Curr. Microbiol. 35: 356-62; Hallmann et al. (1997) 94: 7469-7474; Jakobiak et al. (2004) Protist 155:381-93; Tan et al. (2005) J. Microbiol. 43: 361-365; Steinbrenner et al. (2006) Appl Environ. Microbiol. 72: 7477-7484; Kroth (2007) Methods Mol. Biol. 390: 257-267; U.S. Pat. No. 5,661,017), electroporation (Kjaerulff et al. (1994) Photosynth. Res. 41: 277-283; Iwai et al. (2004) Plant Cell Physiol. 45: 171-5; Ravindran et al. (2006) J. Microbiol. Methods 66: 174-6; Sun et al. (2006) Gene 377: 140-149; Wang et al. (2007) Appl. Microbiol. Biotechnol. 76: 651-657; Chaurasia et al. (2008) J. Microbiol. Methods 73: 133-141; Ludwig et al. (2008) Appl. Microbiol. Biotechnol. 78: 729-35), laser-mediated transformation, incubation with DNA in the presence of or after pre-treatment with any of poly(amidoamine) dendrimers (Pasupathy et al. (2008) Biotechnol. J. 3: 1078-82), polyethylene glycol (Ohnuma et al. (2008) Plant Cell Physiol. 49: 117-120), cationic lipids (Muradawa et al. (2008) J. Biosci. Bioeng. 105: 77-80), dextran, calcium phosphate, and/or calcium chloride (Mendez-Alvarez et al. (1994) J. Bacteriol. 176: 7395-7397), optionally after treatment of the cells with cell wall-degrading enzymes (Perrone et al. (1998) Mol. Biol. Cell 9: 3351-3365), or the like, or combinations thereof. Agrobacterium-mediated transformation can additionally or alternately be performed on algal cells, for example after removing or wounding the algal cell wall (e.g., PCT Publication No. WO 2000/62601; Kumar et al. (2004) Plant Sci. 166: 731-738). Biolistic methods are particularly successful for transformation of the chloroplasts of plant and eukaryotic algal species (see, for example, Ramesh et al. (2004) Methods Mol. Biol. 274: 355-307; Doestch et al. (2001) Curr. Genet. 39: 49-60; U.S. Pat. No. 7,294,506; PCT Publication No. WO 2003/091413; PCT Publication No. WO 2005/005643; and PCT Publication No. WO 2007/133558, all incorporated herein by reference in their entireties).

For optimal expression of a recombinant protein, in many instances it can be beneficial to employ coding sequences producing mRNA with codons preferentially used by the host cell to be transformed. Thus, for an enhanced expression of transgenes, the codon usage of the transgene can be matched with the specific codon bias of the organism in which the transgene is desired to be expressed. For example, methods of recoding genes for expression in microalgae are described in U.S. Pat. No. 7,135,290. The precise mechanisms underlying this effect are believed to be many, but can include the proper balancing of available aminoacylated tRNA pools with proteins being synthesized in the cell, coupled with more efficient translation of the transgenic messenger RNA (mRNA) when this need is met. In some embodiments, only a portion of the codons can be changed to reflect a preferred codon usage of a host microorganism, and in some embodiments, one or more codons can be changed to codons not necessarily the most preferred codon of the host microorganism encoding a particular amino acid. Additional information for codon optimization is available, e.g., at the codon usage database of GenBank. Accordingly, in some embodiments recombinant microorganisms can be transformed with an isolated nucleic acid molecule including a nucleic acid sequence codon-optimized for expression in the recombinant microorganism.

In some embodiments, recombinant microorganisms can be transformed with an isolated nucleic acid molecule including a nucleic acid sequence operably linked to one or more expression control elements. For example, in some preferred embodiments, a gene (such as a gene encoding a chlorophyllase), can be cloned into an expression vector for transformation into a fungus, an alga, or a photosynthetic or non-photosynthetic bacterium. The vector can include sequences promoting expression of the transgene of interest (e.g., the chlorophyllase gene), such as a promoter, and may optionally include, for expression in eukaryotic cells, an intron sequence, a sequence having a polyadenylation signal, or the like, or combinations thereof. Alternatively, if the vector does not contain a promoter in operable linkage with the gene of interest, the gene can be transformed into the cells such that it becomes operably linked to an endogenous promoter, e.g., by homologous recombination, site specific integration, and/or vector integration.

Additionally or alternatively, the vector introduced in to a microorganism can include a promoter or transcriptional enhancer sequence not in operable linkage with a gene of interest, where the promoter or enhancer can be positioned next to one or more sequences for directing the promoter to the chromosomal locus of a gene for producing fatty acids (e.g., an endogenous chlorophyllase gene). For example, sequences for homologous recombination or site-specific recombination can be engineered to flank a transcriptional regulatory sequence in a transformation vector, such that, following transformation into the cells, the regulatory sequence can integrate into the host chromosome and can become operably linked to an endogenous gene, e.g., by homologous recombination, site specific integration, and/or vector integration.

Vectors designed for expression of a gene in microalgae can alternatively or in addition include a promoter active in microalgae operably linked to the exogenous gene being introduced. A variety of gene promoters and terminators functioning in green algae can be utilized in expression vectors, including, but not limited to, promoters and/or terminators from Chlamydomonas and other algae (see, for example, Abe et al. (2008) Plant Cell Physiol, 49: 625-632), promoters and/or terminators from viruses, synthetic promoters and/or terminators, or the like, or combinations thereof.

For transformation of diatoms, a variety of gene promoters that function in diatoms can be utilized in these expression vectors, including, but not limited to: 1) promoters from Thalassiosira and other heterokont algae, promoters from viruses, synthetic promoters, or the like, or combinations thereof. Promoters from Thalassiosira pseudonana and/or Phaeodactylum tricornutum that could be suitable for use in expression vectors can include an alpha-tubulin promoter, a beta-tubulin promoter, an actin promoter, or a combination thereof. The terminators associated with these genes, other diatom genes, and/or particular heterologous genes can be used to stop transcription and/or provide the appropriate signal, e.g., for polyadenylation.

For transformation of cyanobacteria, a variety of promoters functioning in cyanobacteria can be utilized, including, but not limited to, the lac, tac, and trc promoters, as well as derivatives inducible by the addition of isopropyl β-D-1-thiogalactopyranoside (IPTG) such as the trcY or trcE promoter. Other promoters that may be useful can include promoters naturally associated with transposon- or bacterial chromosome-borne antibiotic resistance genes (e.g., neomycin phosphotransferase, chloramphenicol acetyltransferase, spectinomycin adenyltransferase, or the like, or combinations thereof), promoters associated with various heterologous bacterial and native cyanobacterial genes, promoters from viruses and phages, synthetic promoters, or the like, or combinations thereof. Useful promoters isolated from cyanobacteria can include, but are not limited to, the following: nrs (nickel-inducible), secA (secretion; controlled by the redox state of the cell), rbc (Rubisco operon), psaAB (PS I reaction center proteins; light regulated), psbA (Dl protein of PSII; light-inducible), and the like, and combinations thereof. In some embodiments, the promoters can be regulated by nitrogen compounds, such as, for example, nar, ntc, nir or nrt promoters. In some embodiments, the promoters can be regulated by phosphate (e.g., pho or pst promoters) or metals (e.g., the nrs promoter (Liu and Curtis (2009) Proc Natl Acad Sciences USA 106: 21550-21554), or can include the petE promoter (Buikema and Haselkorn (2001) Proc Natl Acad Sciences USA 98: 2729-2734)).

Likewise, a wide variety of transcriptional terminators can be used for expression vector construction. Examples of possible terminators can include, but are not limited to, psbA, psaAB, rbc, secA, T7 coat protein, and the like, and combinations thereof.

Transformation vectors can additionally or alternatively include a selectable marker, such as but not limited to a drug resistance gene, an herbicide resistance gene, a metabolic enzyme and/or factor required for survival of the host (for example, an auxotrophic marker), or the like, or a combination thereof. Transformed cells can be optionally selected based upon the ability to grow in the presence of the antibiotic and/or other selectable marker under conditions in which cells lacking the resistance cassette or auxotrophic marker could not grow. Further additionally or alternatively, a non-selectable marker may be present on a vector, such as a gene encoding a fluorescent protein or enzyme that generates a detectable reaction product.

### Antisense Constructs

In some instances, it can be advantageous to express an antisense molecule, or a gene encoding an exogenous and/or heterologous enzyme, such as but not limited to a lipase, at a certain point during the growth of the transgenic host, e.g., to minimize any deleterious effects on the growth of the transgenic organism and/or to maximize production of the fatty acid product of interest. In such instances, one or more exogenous genes, operably linked to a promoter, can be introduced into the transgenic organism. The promoter can be a constitutive or regulatable promoter. The promoter can be, for example, a lac promoter, a tet promoter (e.g., U.S. Pat. No. 5,851,796), a hybrid promoter that includes either or both of portions of a tet or lac promoter, a hormone-responsive promoter (e.g., an ecdysone-responsive promoter, such as described in U.S. Pat. No. 6,379,945), a metallothionien promoter (e.g., U.S. Pat. No. 6,410,828), a pathogenesis-related (PR) promoter that can be responsive to a chemical such as, for example, salicylic acid, ethylene, thiamine, and/or BTH (U.S. Pat. No. 5,689,044), or the like, or some combination thereof. An inducible promoter can also be responsive to light or dark (U.S. Pat. No. 5,750,385, U.S. Pat. No. 5,639,952) or temperature (U.S. Pat. No. 5,447,858; Abe et al. (2008) Plant Cell Physiol. 49: 625-632; Shroda et al. (2000) Plant J. 21: 121-131). The foregoing list is exemplary and not limiting. Alternatively or in addition, the antisense construct can be integrated into the host microorganism's genome, such that the antisense sequence can become operably linked to an endogenous promoter of the host. In particular examples, the promoter can be an endogenous promoter active under the same conditions as the promoter regulating expression of the endogenous gene encoding a polypeptide that can degrade or otherwise inhibit chlorophyllase. In further examples, an antisense construct can include a copy of the same promoter regulating the expression of the target gene in the host microorganism. The promoter sequences can be from any organism, provided that they are functional in the host organism. Inducible promoters can use one or more portions or domains of the aforementioned promoters and/or other inducible promoters fused to at least a portion of a different promoter that can operate in the host organism, e.g., to confer inducibility on a promoter that operates in the host species. For example, an inducible promoter can respond to light, to temperature, to pH, and/or to a chemical such as selected from the group consisting of carbon, cobalt, copper, iron, nickel, nitrogen, and oxygen.

The expression of a gene encoding a polypeptide that can degrade or otherwise inhibit chlorophyllase can be reduced or eliminated by expression of an antisense construct introduced into the photosynthetic microorganism. As used herein, an antisense construct refers particularly to a nucleic acid molecule including a sequence encoding an antisense molecule, i.e., a ribonucleotide sequence having homology to at least a portion of the non-coding strand of a double stranded DNA molecule of a gene encoding a protein (for example, at least 85%, at least 90%, at least 95%, at least 97%, at least 99%, or about 100% identical to at least a portion of the non-coding strand of a gene). Thus, an antisense molecule or “antisense RNA” can be complementary to at least a portion of the sequence of the coding strand of a double stranded DNA molecule encoding a polypeptide. The antisense sequence may be complementary to protein-coding sequences of the targeted gene, and alternatively or in addition, the antisense sequence may be complementary, wholly or in part, to noncoding sequences specified on the transcribed strand of a DNA molecule encoding a protein, for example, a 5′untranslated region (UTR) and/or an intron. Antisense sequences can be at least 85% complementary, e.g., at least 90% or at least 95% complementary, to the target nucleic acid (gene) sequence. Expression of an antisense construct can result in the production of an antisense RNA having substantial or complete identity to at least a portion of a target gene. An antisense construct can include an antisense sequence of at least about twenty nucleotides, for example, at least about 25, at least about 30, at least about 40, at least about 50, at least about 100 or at least about 200 nucleotides, having at least 85%, at least 90%, or at least 95% identity to a sequence complementarity to a transcribed portion of a gene. Additionally and/or alternatively, catalytic RNA molecules or ribozymes can be used in place of antisense RNA. For example, one or more ribozymes can be designed to specifically pair with the target RNA and cleave the phosphodiester backbone to inactivate the target RNA.

### Methods of Increasing Photosynthetic Efficiency

Without being be bound by theory, it has been hypothesized that overall photosynthetic efficiency of the culture could be improved if each microorganism had a smaller light-harvesting antenna, such that the organisms/cells otherwise in the path of photons on the way to other organisms/cells would be unable to capture excess photons, which photons would then be permitted to pass deeper into the culture where they could reach cells that would be otherwise light-deprived. Melis (2009) Plant Sci. 177:272-80. Therefore, methods of increasing the photosynthetic efficiency of a culture of algal cells are disclosed herein. These methods can comprise the step of culturing algal cells that can overexpress a chlorophyllase. In certain embodiments, these cultures can be cultivated in a pond or bioreactor. The algal cells overexpressing a chlorophyllase can also overexpress one or more additional genes as described elsewhere herein. The algal cells overexpressing a chlorophyllase can additionally or alternatively be attenuated with regard to one or more genes, as described elsewhere herein.

### Methods of Producing Biomass or One or More Biomolecules

Methods of producing biomass generally or of selectively producing at least one particular biomolecule are also described herein. Photosynthetic organisms perform photosynthesis, absorb carbon dioxide (CO₂), and convert it to biomass. When that biomass is burned, it can be possible to recover heat energy. Alternatively, biomass can be added to food or to animal feed, and/or can be converted to organic molecules (e.g., alcohols, through fermentation) that can be fuel or chemical feedstocks. Therefore, methods of producing biomass can comprise culturing the photosynthetic microorganisms described herein and isolating biomass or at least one biomolecule from the culture. The photosynthetic microorganisms used for production of biomass or a biomolecule can advantageously overexpress chlorophyllase. In certain embodiments, the microorganism can overexpress both chlorophyllase and at least one additional enzyme selected from the group consisting of magnesium dechelatase, pheophorbide a oxygenase, pheophorbidase, and red chlorophyll catabolite reductase. The recombinant photosynthetic microorganisms described herein can thus exhibit reduced chlorophyll a content, relative to an otherwise identical control microorganism that does not overexpress chlorophyllase. In some embodiments, the total chlorophyll a content can be at least 5%, at least 10%, at least 15%, at least 20%, at least 25%, at least 30%, at least 40%, at least 50%, at least 60%, at least 70%, at least 80%, or at least 90% reduced, relative to the control microorganism. Chlorophyll a content can be measured by a variety of methods known in the art, such as extraction in methanol and spectrophotometric quantification.

The photosynthetic microorganism can be cultured as an actively mixed culture, for example in a pond or photobioreactor. For example, the photosynthetic microorganism can be cultured in a pond having a depth of at least 3 cm, at least 5 cm, or at least 10 cm, or a photobioreactor having a light path of at least 3 cm, at least 5 cm, or at least 10 cm. As used herein “pond” means any open body of water, whether naturally-occurring or man-made, including ponds, canals, trenches, lagoons, channels, or raceways. The pond or bioreactor can include at least one active mixing device, such as a paddlewheel, pump, propeller, fluid injection system, sparger, or any combination thereof, optionally in combination with at least one passive mixing device. Further additionally or alternatively, the photosynthetic microorganism can be cultured in a volume of at least 20 liters of culture medium.

In some embodiments, the amount of biomass or of a biomolecule produced by the culture can be at least 10%, for example at least 15%, at least 20%, or at least 25%, greater than the amount of a biomolecule produced by an identical culture of a control (or wild type) microorganism. Additionally or alternatively, the photosynthetic microorganism can be cultured phototrophically and/or under intermittent light conditions, e.g., in an actively mixed culture, optionally under natural light.

In some aspects, the microorganisms can be cultured in a suitable culture medium, which in certain examples can be a culture medium not including a substantial amount of a reduced carbon source, such that the cells are cultured photoautotrophically. Additionally, the culture medium can include inorganic carbon as substantially the sole source of carbon for production of the biomolecule.

Culturing refers to the intentional fostering of growth (e.g., increases in cell size, cellular contents, and/or cellular activity, e.g., biomolecule synthesis) and/or propagation (e.g., increases in cell numbers via mitosis) of one or more cells by use of selected and/or controlled conditions. The combination of both growth and propagation may be termed proliferation. Non-limiting examples of selected and/or controlled conditions can include the use of a defined medium (with known characteristics such as pH, ionic strength, and/or carbon source), specified temperature, oxygen tension, carbon dioxide levels, growth in a bioreactor, mixing of the culture, or the like, or combinations thereof. In some embodiments, the microorganism can be grown heterotrophically or mixotrophically, using both light and a reduced carbon source. In certain embodiments, the microorganism can preferably be cultured phototrophically. When growing or propagating phototrophically, the microorganism can advantageously use light as an energy source. An inorganic carbon source, such as CO₂ or bicarbonate, can be used for synthesis of biomolecules by the microorganism. “Inorganic carbon”, as used herein, includes carbon-containing compounds or molecules that cannot be used as a sustainable energy source by an organism. Typically “inorganic carbon” can be in the form of CO₂ (carbon dioxide), carbonic acid, bicarbonate salts, carbonate salts, hydrogen carbonate salts, or the like, or combinations thereof, which cannot be further oxidized for sustainable energy nor used as a source of reducing power by organisms. If an organic carbon molecule or compound is provided in the culture medium of a microorganism grown phototrophically, it generally cannot be taken up and/or metabolized by the cell for energy and/or typically is not present in an amount sufficient to provide sustainable energy for the growth of the cell culture.

A source of inorganic carbon (such as, but not limited to, CO₂, bicarbonate, carbonate salts, and the like), including, but not limited to, air, CO₂-enriched air, flue gas, or the like, or combinations thereof, can be supplied to the culture. When supplying flue gas and/or other sources of inorganic that may contain CO in addition to CO₂, it may be necessary to pre-treat such sources such that the CO level introduced into the (photo)bioreactor do not constitute a dangerous and/or lethal dose vis-à-vis the growth and/or survival of the microorganisms.

In some embodiments, a microorganism that produces one or more free fatty acids can be cultured in a medium including an increased concentration of a metal (typically provided as a salt and/or in an ionic form) such as, for example, sodium, potassium, magnesium, calcium, iron, or the like, or combinations thereof (particularly multivalent metals, such as magnesium, calcium, and/or iron). For example, a medium used for growing microorganisms that produce and release into the culture medium one or more free fatty acids can include at least 2-fold, for example at least 3-fold, at least 4-fold, at least 5-fold, at least 6-fold, at least 7-fold, at least 8-fold, at least 9-fold, at least 10-fold, between 2-fold and 10-fold, and/or between 10-fold and 100-fold the amount of metal (e.g., calcium and/or magnesium) as compared to a standard medium. Other details regarding the use of a soap-forming ion source in the algal medium are described in U.S. Pat. No. 9,187,726, which is incorporated herein by reference.

Culturing of photosynthetic microorganisms can be performed under various conditions, such as under a light/dark cycle, and/or under natural light. In some embodiments, light/dark cycle refers to providing and removing (e.g., switching on and off) the light over a predetermined period, for example, a light dark cycle can be 12 hours of light followed by 12 hours of darkness or 14 hours of light followed by 10 hours of darkness. Alternatively or in addition, the light/dark cycle can be a natural light/dark cycle based on day-length, where the sun is the light source. Natural light can optionally be supplemented by artificial light. In some culture systems, the light period of a culture grown under natural light can be extended by the inclusion of one or more artificial light sources. When the cultures are grown under artificial light, the light can be of any amount of photosynthetically active radiation, for example at least 50, at least 100, at least 250, at least 500, at least 750, at least 1000, at least 1500, at least 2000, at least 3000, at least 4000, at least 5000, at least 7500, or at least 10000 μEm⁻² s⁻¹.

The depth of a pond or light path of a photobioreactor can play a factor in the amount of mixing needed to achieve a desired level of turbulence. For example, the depth of the growth pond can have a substantial impact on the Reynolds number for the pond. As an illustrative example, if the depth of the pond is reduced from 30 cm to 10 cm, the Reynolds number of such a pond can increase from about 1000 to about 3000.

Biomass of the microorganism culture can be recovered by harvesting the microorganism from the medium, for example, by filtering, settling, centrifugation, or combinations thereof. In biomass production embodiments, the amount of the biomass produced and/or recovered by the method described herein, measured as ash-free dry weight (AFDW), can advantageously be at least about 0.05 g per liter of culture, for example at least about 0.1 g, at least about 0.2 g, at least about 0.3 g, at least about 0.4 g, at least about 0.5 g, at least about 0.6 g, at least about 0.7 g per liter of culture, at least about 1 g per liter of culture, at least about 1.5 g per liter of culture, at least about 2 g per liter of culture, at least about 2.5 g per liter of culture, or at least about 5 g per liter of culture. Although many times the goal can be to produce and/or recover as much biomass as possible, in some instances the amount of the biomass produced and/or recovered by the method described herein, measured as ash free dry weigh (AFDW) can be limited to about 15 g or less per liter of culture, for example about 12 g or less per liter of culture, about 10 g or less per liter of culture, about 5 g or less per liter of culture, about 2 g or less per liter of culture, about 1 g or less per liter of culture, or about 0.5 g or less per liter of culture.

The dry weight (DW) and ash-free dry weight (AFDW) can be calculated according to the formulas:

$$DW ( g \text{/} l ) = \frac{( ovenweight ( g ) - filterweight ( g ) ) * 1000 ( ml \text{/} l )}{samplevolume ( ml )}$$

$$AFDW ( g \text{/} l ) = DW ( g \text{/} l ) - ( \frac{( furnaceweight ( g ) - filterweight ( g ) )}{samplevolume ( ml )} * 1000 ( ml \text{/} l ) )$$

where “ovenweight” is the weight of the sample after drying in the oven, and “furnaceweight” is the weight of the same sample after combusting in the muffle furnace.

By way of representative procedure for measuring biomass, ˜25 to ˜35 mL of removed sample of each culture can be transferred to a filtration assembly that includes a side arm flask fitted with a stopper, funnel, and screen for supporting a filter held with a clamp. A pre-weighed Whatman ˜47 mm GF/F glass microfiber filter can be positioned over the screen. The sample can be pipetted onto the surface of the filter, and a vacuum (about ˜5-10 psi) applied via the side arm of the flask. Once all the liquid passes through the filter, the sides of the funnel can be rinsed with ˜9-12 mL distilled water to bring down any cells that may have stuck to the side of the funnel. The rinsing step can be repeated, e.g., twice. Once the filtration is finished, the filter can be removed from the base, e.g., with forceps. The filter can be placed in an appropriate vessel, such as a pre-weighed aluminum weighing boat, and then the samples placed in a ˜105° C. drying oven until the weight is constant, e.g. at least four hours. The dried samples can then be placed in a desiccator to cool, and then the weigh boat plus filter weighed. Dry weight is calculated as:

$$DW ( g \text{/} l ) = \frac{( ovenweight ( g ) - vialweight ( g ) ) * 1000 ( ml \text{/} l )}{samplevolume ( ml )}$$

Samples can then be placed into a muffle furnace heated to ˜550° C. for ˜1 hour. The samples can then be removed (using tongs) and transferred to the desiccator to cool to room temperature (˜20-25° C.). When the samples have cooled, they can be weighed using the same analytical balance used to weigh the dry samples.

Ash Free Dry Weight (in g/l) is calculated as follows:

$$AFDW ( g \text{/} l ) = DW ( g \text{/} l ) - ( \frac{( furnaceweight ( g ) - vialweight ( g ) )}{samplevolume ( ml )} * 1000 ( ml \text{/} l ) )$$

Biomass can be used in any of a number of ways. For example, it can be processed for use as a biofuel by generating syngas from the biomass; it can be supplied to an anaerobic digester for production of one or more alcohols; and/or it can be extracted to provide algal lipids, such as but not limited to monoglycerides, diglycerides, or triglycerides, fatty acid alkyl esters, fatty acids, and/or fatty acid derivatives.

In some embodiments, fatty acids and fatty acid derivatives can be recovered from culture by recovery means known to those of ordinary skill in the art, such as by whole culture extraction, for example, using organic solvents. In some cases, recovery of fatty acids or fatty acid derivatives (such as fatty acid esters) can be enhanced by homogenization of the cells. When fatty acids are sufficiently released from the microorganisms into the culture medium, the recovery method can be adapted to efficiently recover only the released fatty acids, only the fatty acids produced and stored within the microorganisms, or both the produced and released fatty acids.

In further embodiments, products such as but not limited to free fatty acids and fatty acid derivatives secreted/released into the culture medium by the recombinant microorganisms described above can be recovered in a variety of ways. A straightforward isolation method, e.g., by partition using immiscible solvents, may be employed. Additionally or alternatively, particulate adsorbents can be employed. These can include lipophilic particulates and/or ion exchange resins, depending on the design of the recovery method. They may be circulating in the separated medium and then collected, and/or the medium may be passed over a fixed bed column, for example a chromatographic column, containing these particulates. The fatty acids can then be eluted from the particulate adsorbents, e.g., by the use of an appropriate solvent. In such circumstances, one isolation method can include carrying out evaporation of the solvent, followed by further processing of the isolated fatty acids and lipids, to yield chemicals and/or fuels that can be used for a variety of commercial purposes.

The microorganisms according to some embodiments can produce free fatty acids and fatty acid derivatives in an amount greater than the amount of free fatty acids and fatty acid derivatives produced by a control strain that has not overexpressed chlorophyllase, but which was grown under identical conditions.

### Further Embodiments

Additionally or alternately, the present invention described herein can include one or more of the following embodiments.

### Embodiment 1

A recombinant microalga comprising a heterologous expression construct, wherein the heterologous expression construct comprises: an inducible promoter operably linked to a nucleotide sequence encoding a chlorophyllase (such as comprising an amino acid sequence having at least 85% identity to SEQ ID NO:1); optionally a nucleotide sequence encoding a pheophorbidase (such as having at least 60%, at least 65%, at least 70%, at least 75%, at least 80%, or at least 85% identity to SEQ ID NO: 2); optionally a nucleotide sequence encoding a pheophorbide a oxygenase (such as having at least 60%, at least 65%, at least 70%, at least 75%, at least 80%, or at least 85% identity to SEQ ID NO:3), optionally a nucleotide sequence encoding a red chlorophyll catabolite reductase (such as having at least 60%, at least 65%, at least 70%, at least 75%, at least 80%, or at least 85% identity to SEQ ID NO:4); and optionally a magnesium dechetalase.

### Embodiment 2

The recombinant microalga of Embodiment 1, wherein at least one of the nucleotide sequence encoding the pheophorbidase, the nucleotide sequence encoding the pheophorbide a oxygenase, and the nucleotide sequence encoding the red chlorophyll catabolite reductase is present on the same expression construct with the nucleotide sequence encoding the chlorophyllase and under the control of the same inducible promoter as the nucleotide sequence encoding the chlorophyllase.

### Embodiment 3

The recombinant microalga of Embodiment 1 or Embodiment 2, wherein the inducible promoter is a light inducible promoter, a temperature sensitive promoter, a pH sensitive promoter, or a promoter responsive to a chemical selected from the group consisting of carbon, cobalt, copper, iron, nickel, nitrogen, and oxygen.

### Embodiment 4

The recombinant microalga of any one of the previous Embodiments, wherein the recombinant microalga has a total chlorophyll a content of at least 10% less (for example at least 15% less, at least 20% less, at least 25% less, at least 30% less, at least 40% less, or at least 50% less) than the chlorophyll a content of an otherwise identical control microalga that does not comprise the heterologous expression construct.

### Embodiment 5

The recombinant microalga of any one of the previous Embodiments, wherein the recombinant microalga is a species belonging to a genus selected from the group consisting of Achnanthes, Amphiprora, Amphora, Ankistrodesmus, Asteromonas, Boekelovia, Bolidomonas, Borodinella, Botrydium, Botryococcus, Bracteococcus, Chaetoceros, Carteria, Chlamydomonas, Chlorococcum, Chlorogonium, Chlorella, Chroomonas, Chrysosphaera, Cricosphaera, Crypthecodinium, Cryptomonas, Cyclotella, Dunaliella, Elhpsoidon, Emiliania, Eremosphaera, Ernodesmius, Euglena, Eustigmatos, Franceia, Fragilaria, Gloeothamnion, Haematococcus, Halocafeteria, Heterosigma, Hymenomonas, Isochrysis, Lepocinclis, Micractinium, Monoraphidium, Nannochloris, Nannochloropsis, Navicula, Neochloris, Nephrochloris, Nephroselmis, Nitzschia, Ochromonas, Oedogonium, Oocystis, Ostreococcus, Pavlova, Parachlorella, Pascheria, Pelagomonas, Phaeodactylum, Phagus, Picochlorum, Platymonas, Pleurochrysis, Pleurococcus, Prototheca, Pseudochlorella, Pseudoneochloris, Pseudostaurastrum, Pyramimonas, Pyrobotrys, Scenedesmus, Schizochlamydella, Skeletonema, Spyrogyra, Stichococcus, Tetraselmis, Thalassiosira, Tribonema, Vaucheria, Viridiella, Vischeria, and Volvox.

### Embodiment 6

A method for increasing photosynthetic efficiency of a microalgal culture, the method comprising culturing the recombinant microalga of any one of the previous embodiments in a culture medium.

### Embodiment 7

A method for producing biomass or a biomolecule, the method comprising culturing the recombinant microalga of any one of Embodiments 1-5 in a culture medium.

### Embodiment 8

The method of Embodiment 6 or Embodiment 7, wherein the culture experiences active mixing.

### Embodiment 9

The method of any one of Embodiments 6-8, wherein the culture is illuminated with artificial light having an intensity greater than 1000 μEm⁻² s⁻¹.

### Embodiment 10

The method of any one of Embodiments 6-9, wherein the illumination simulates a diel cycle.

### Embodiment 11

The method of any one of Embodiments 6-10, wherein the culture is illuminated with natural light.

### Embodiment 12

The method of any one of Embodiments 6-11, wherein the culture is cultured outdoors under solar illumination.

Further additionally or alternately, there can be a method according to any one of the preceding method embodiments, wherein the medium used for culturing the fatty acid-producing organism can include an increased concentration of a soap-forming ion source (e.g., an inorganic soap-forming ion source, a metal ion source, a multivalent metal ion source, a divalent metal ion source, or some combination thereof, such as sodium, potassium, magnesium, calcium, iron, or combinations thereof, particularly multivalent metals, such as magnesium, calcium, and/or iron), with respect to a standard medium formulation (e.g., standard BG-11 medium) or a modified medium (e.g., ATCC Medium 854 or ATCC Medium 617), which increased concentration can optionally be at least about 0.5 mM (e.g., between about 0.5 mM and about 1 mM, between about 1 mM and about 2 mM, between about 2 mM and about 5 mM, between about 5 mM and about 10 mM, between about 10 mM and about 25 mM, and/or greater than 25 mM) and/or can optionally but preferably be at least 2-fold (e.g., at least 3-fold, at least 4-fold, at least 5-fold, at least 6-fold, at least 7-fold, at least 8-fold, at least 9-fold, at least 10-fold, between 2-fold and 10-fold, and/or between 10-fold and 100-fold) as compared to said standard/modified medium.

Without further description, it is believed that one of ordinary skill in the art can, using the preceding description and the following illustrative examples, make and utilize the microorganisms described herein and practice the methods disclosed herein.

1. A recombinant microalga comprising a heterologous expression construct, wherein the heterologous expression construct comprises an inducible promoter operably linked to a nucleotide sequence encoding a chlorophyllase.

2. The recombinant microalga of claim 1, wherein the chlorophyllase comprises an amino acid sequence having at least 85% identity to SEQ ID NO:1.

3. The recombinant microalga of claim 1, further comprising a nucleotide sequence encoding a pheophorbidase.

4. The recombinant microalga of claim 3, wherein the pheophorbidase has at least 85% identity to SEQ ID NO: 2.

5. The recombinant microalga of claim 1, further comprising a nucleotide sequence encoding a pheophorbide a oxygenase.

6. The recombinant microalga of claim 5, wherein the pheophorbide a oxygenase has at least 85% identity to SEQ ID NO:3.

7. The recombinant microalga of claim 1, further comprising a nucleotide sequence encoding a red chlorophyll catabolite reductase.

8. The recombinant microalga of claim 7, wherein the red chlorophyll catabolite reductase has at least 85% identity to SEQ ID NO:4.

9. The recombinant microalga of claim 1, further comprising a pheophorbidase, a pheophorbide a oxygenase, and a red chlorophyll catabolite reductase.

10. The recombinant microalga of claim 2, further comprising:
a pheophorbidase having at least 85% identity to SEQ ID NO: 2;
a pheophorbide a oxygenase having at least 85% identity to SEQ ID NO:3; and
a red chlorophyll catabolite reductase having at least 85% identity to SEQ ID NO:4.

11. The recombinant microalga of claim 10, wherein at least one of the nucleotide sequence encoding the pheophorbidase, the nucleotide sequence encoding the pheophorbide a oxygenase, and the nucleotide sequence encoding the red chlorophyll catabolite reductase is present on the same expression construct with the nucleotide sequence encoding the chlorophyllase and under the control of the same inducible promoter as the nucleotide sequence encoding the chlorophyllase.

12. The recombinant microalga of claim 1, wherein the inducible promoter is a light inducible promoter, a temperature sensitive promoter, a pH sensitive promoter, or a promoter responsive to a chemical selected from the group consisting of carbon, cobalt, copper, iron, nickel, nitrogen, and oxygen.

13. The recombinant microalga of claim 1, wherein the recombinant microalga has a total chlorophyll a content of at least 10% less than the chlorophyll a content of an otherwise identical control microalga that does not comprise the heterologous expression construct.

14. The recombinant microalga of claim 1, wherein the recombinant microalga is a species belonging to a genus selected from the group consisting of Achnanthes, Amphiprora, Amphora, Ankistrodesmus, Asteromonas, Boekelovia, Bolidomonas, Borodinella, Botrydium, Botryococcus, Bracteococcus, Chaetoceros, Carteria, Chlamydomonas, Chlorococcum, Chlorogonium, Chlorella, Chroomonas, Chrysosphaera, Cricosphaera, Crypthecodinium, Cryptomonas, Cyclotella, Dunaliella, Elhpsoidon, Emiliania, Eremosphaera, Ernodesmius, Euglena, Eustigmatos, Franceia, Fragilaria, Gloeothamnion, Haematococcus, Halocafeteria, Heterosigma, Hymenomonas, Isochrysis, Lepocinclis, Micractinium, Monoraphidium, Nannochloris, Nannochloropsis, Navicula, Neochloris, Nephrochloris, Nephroselmis, Nitzschia, Ochromonas, Oedogonium, Oocystis, Ostreococcus, Pavlova, Parachlorella, Pascheria, Pelagomonas, Phaeodactylum, Phagus, Picochlorum, Platymonas, Pleurochrysis, Pleurococcus, Prototheca, Pseudochlorella, Pseudoneochloris, Pseudostaurastrum, Pyramimonas, Pyrobotrys, Scenedesmus, Schizochlamydella, Skeletonema, Spyrogyra, Stichococcus, Tetraselmis, Thalassiosira, Tribonema, Vaucheria, Viridiella, Vischeria, and Volvox.

15. A method for increasing photosynthetic efficiency of a microalgal culture, the method comprising culturing the recombinant microalga of claim 1 in a culture medium.

16. The method of claim 15, wherein the culture experiences active mixing.

17. The method of claim 15, wherein the culture is illuminated with artificial light having an intensity greater than 1000 μEm⁻² s⁻¹.

18. The method of claim 17, wherein the illumination simulates a diel cycle.

19. The method of claim 15, wherein the culture is illuminated with natural light.

20. The method of claim 19, wherein the culture is cultured outdoors under solar illumination.

21. The method of claim 15, wherein the chlorophyllase comprises an amino acid sequence having at least 85% identity to SEQ ID NO:1.

22. The method of claim 21, wherein the recombinant microalga further comprises at least one additional enzyme selected from the group consisting of:
a pheophorbidase having at least 85% identity to SEQ ID NO: 2;
a pheophorbide a oxygenase having at least 85% identity to SEQ ID NO:3; and
a red chlorophyll catabolite reductase having at least 85% identity to SEQ ID NO:4.

23. The method of claim 15, wherein the inducible promoter is a light inducible promoter, a temperature sensitive promoter, a pH sensitive promoter, or a promoter responsive to a chemical selected from the group consisting of carbon, cobalt, copper, iron, nickel, nitrogen, and oxygen.

24. The method of claim 15, wherein the recombinant microalga has a total chlorophyll a content of at least 10% less than the chlorophyll a content of an otherwise identical control microalga that does not comprise the heterologous expression construct.

25. The method of claim 15, wherein the recombinant microalga is a species belonging to a genus selected from the group consisting of Achnanthes, Amphiprora, Amphora, Ankistrodesmus, Asteromonas, Boekelovia, Bolidomonas, Borodinella, Botrydium, Botryococcus, Bracteococcus, Chaetoceros, Carteria, Chlamydomonas, Chlorococcum, Chlorogonium, Chlorella, Chroomonas, Chrysosphaera, Cricosphaera, Crypthecodinium, Cryptomonas, Cyclotella, Dunaliella, Elhpsoidon, Emiliania, Eremosphaera, Ernodesmius, Euglena, Eustigmatos, Franceia, Fragilaria, Gloeothamnion, Haematococcus, Halocafeteria, Heterosigma, Hymenomonas, Isochrysis, Lepocinclis, Micractinium, Monoraphidium, Nannochloris, Nannochloropsis, Navicula, Neochloris, Nephrochloris, Nephroselmis, Nitzschia, Ochromonas, Oedogonium, Oocystis, Ostreococcus, Pavlova, Parachlorella, Pascheria, Pelagomonas, Phaeodactylum, Phagus, Picochlorum, Platymonas, Pleurochrysis, Pleurococcus, Prototheca, Pseudochlorella, Pseudoneochloris, Pseudostaurastrum, Pyramimonas, Pyrobotrys, Scenedesmus, Schizochlamydella, Skeletonema, Spyrogyra, Stichococcus, Tetraselmis, Thalassiosira, Tribonema, Vaucheria, Viridiella, Vischeria, and Volvox.

26. A method for producing biomass or a biomolecule, the method comprising culturing the recombinant microalga of claim 1 in a culture medium.

27. The method of claim 26, wherein the culture experiences active mixing.

28. The method of claim 26, wherein the culture is illuminated with artificial light having an intensity greater than 1000 μEm⁻² s⁻¹.

29. The method of claim 28, wherein the illumination simulates a diel cycle.

30. The method of claim 26, wherein the culture is illuminated with natural light.

31. The method of claim 30, wherein the culture is cultured outdoors under solar illumination.

32. The method of claim 26, wherein the chlorophyllase comprises an amino acid sequence having at least 85% identity to SEQ ID NO:1.

33. The method of claim 32, wherein the recombinant microalga further comprises at least one additional enzyme selected from the group consisting of:
a pheophorbidase having at least 85% identity to SEQ ID NO: 2;
a pheophorbide a oxygenase having at least 85% identity to SEQ ID NO:3; and
a red chlorophyll catabolite reductase having at least 85% identity to SEQ ID NO:4.

34. The method of claim 26, wherein the inducible promoter is a light inducible promoter, a temperature sensitive promoter, a pH sensitive promoter, or a promoter responsive to a chemical selected from the group consisting of carbon, cobalt, copper, iron, nickel, nitrogen, and oxygen.

35. The method of claim 26, wherein the recombinant microalga has a total chlorophyll a content of at least 10% less than the chlorophyll a content of an otherwise identical control microalga that does not comprise the heterologous expression construct.

36. The method of claim 26, wherein the recombinant microalga is a species belonging to a genus selected from the group consisting of Achnanthes, Amphiprora, Amphora, Ankistrodesmus, Asteromonas, Boekelovia, Bolidomonas, Borodinella, Botrydium, Botryococcus, Bracteococcus, Chaetoceros, Carteria, Chlamydomonas, Chlorococcum, Chlorogonium, Chlorella, Chroomonas, Chrysosphaera, Cricosphaera, Crypthecodinium, Cryptomonas, Cyclotella, Dunaliella, Elhpsoidon, Emiliania, Eremosphaera, Ernodesmius, Euglena, Eustigmatos, Franceia, Fragilaria, Gloeothamnion, Haematococcus, Halocafeteria, Heterosigma, Hymenomonas, Isochrysis, Lepocinclis, Micractinium, Monoraphidium, Nannochloris, Nannochloropsis, Navicula, Neochloris, Nephrochloris, Nephroselmis, Nitzschia, Ochromonas, Oedogonium, Oocystis, Ostreococcus, Pavlova, Parachlorella, Pascheria, Pelagomonas, Phaeodactylum, Phagus, Picochlorum, Platymonas, Pleurochrysis, Pleurococcus, Prototheca, Pseudochlorella, Pseudoneochloris, Pseudostaurastrum, Pyramimonas, Pyrobotrys, Scenedesmus, Schizochlamydella, Skeletonema, Spyrogyra, Stichococcus, Tetraselmis, Thalassiosira, Tribonema, Vaucheria, Viridiella, Vischeria, and Volvox.
